(19) United States
(12) Patent Application Publication
(10) Pub. No.: US 2008/0287780 A1
(43) Pub. Date: Nov. 20, 2008

(54) INTEGRAL BASED PARAMETER IDENTIFICATION APPLIED TO THREE DIMENSIONAL TISSUE STIFFNESS RECONSTRUCTION IN A DIGITAL IMAGE-BASED ELASTO-TOMOGRAPHY SYSTEM

(76) Inventors: James Geoffrey Chase, Fendalton (NZ); Christopher Eric Hann, Rangiora (NZ)

Correspondence Address: HISCOCK & BARCLAY, LLP, 2000 HSBC PLAZA, 100 Chestnut Street, ROCHESTER, NY, 14604-2404, US

(21) Appl. No.: 11/749,633

(22) Filed: May 16, 2007

(51) Int. Cl. A61B 6/03

(52) U.S. Cl. 600/425

(57) ABSTRACT

The invention includes a method and a system for obtaining accurate patient specific parameter identification at high resolution with a minimal amount of computation as applied to breast tissue stiffness reconstruction from a digital image-based elasto-tomography system. The method includes the steps of formulating the differential equation model describing tissue motion in terms of integrals of breast tissue displacement data that is measured using calibrated digital cameras; setting up a system of linear equations in the space varying tissue stiffness parameters; and solving by linear least squares to obtain the unique patient specific breast tissue stiffness distribution.

### FIELD OF THE INVENTION

The invention relates generally to the field of breast cancer screening, and in particular to a technique of Breast tissue Stiffness Reconstruction from breast surface displacement data in a digital image-based elasto-tomography system.

### BACKGROUND OF THE INVENTION

Breast cancer is a significant health problem in both developed and developing countries. It is estimated that each year the disease is diagnosed in over 1,000,000 women worldwide and is the cause of death in over 400,000 women. There are many treatment options available, including surgery, chemotherapy, radiation therapy, and hormonal therapy. These treatments are significantly more effective in reducing the mortality of the disease with early detection through breast cancer screening programmes.

The standard method for detection of breast cancer is mammography. However mammography can cause significant patient discomfort and requires radiation exposure. Furthermore there are often variable results and inconsistencies in reading and interpreting the images of breast tissue from the X-Ray machine especially for smaller tumour sizes of the order of 1-5 mm.

Digital Image based Elasto-tomography is an emerging technology for non-invasive breast cancer screening without the requirement of radiation. As used herein, Digital Image-based Elasto-Tomography system will be referred to as a DIET system. The DIET system uses digital imaging of an actuated breast surface to determine tissue surface motion. It then reconstructs the three-dimensional internal tissue stiffness distribution from that motion. Regions of high stiffness suggest cancer since cancerous tissue is between 3 and 10 times stiffer than healthy tissue in the breast. This approach eliminates the need for X-Rays and excessive, potentially painful compression of the breast as required in a mammogram. Hence, screening could start much younger and might enjoy greater compliance. Presently, there are other elasto-tomographic methods based on magnetic resonance and ultrasound modalities. Both methods are capable of measuring the tissue elasticity and are undergoing rapid development across the globe. However, they are also costly in terms of equipment and take significant time to use. They are therefore limited for practical screening applications.

The DIET system, in contrast, is silicon based and is thus potentially low cost and portable, so the technology could be used in any medical centre, particularly in remote areas. In addition, the use of silicon technology ensures that as it improves and scales upward in capability so will the DIET system performance. This scalability of performance is not true for X-Ray or ultrasound based approaches.

The DIET system relies on a fast and accurate breast tissue stiffness reconstruction from breast surface displacement data. Furthermore the stiffness distribution must be high resolution to ensure small tumours are not missed. Therefore, there exists a need in the art for very accurate patient-specific parameter identification that can deal with the unique requirements of a DIET system. In addition for clinical effectiveness, the stiffness reconstruction must be done with a minimal amount of computation.

### SUMMARY OF THE INVENTION

The present invention is directed towards overcoming the problem of very high resolution patient specific parameter identification with a minimal amount of computation in connection with the DIET system; comprising a patient bed, an actuator to induce oscillation in a tissue, such as breast tissue, an array of digital cameras and computer software for processing images of the breast surface and transforming into measured motion, and computer software for converting measured motion into a three-dimensional distribution of stiffness of the breast.

Briefly summarized, according to one aspect of the invention a method for accurately identifying space varying patient specific parameters in a non-linear differential equation model as applied to tissue reconstruction from such a DIET system as described above comprises the steps of providing measured tissue displacement data; formulating a differential equation model describing tissue motion in terms of integrals of the measured tissue displacement data; setting up a system of linear equations in the space varying tissue stiffness parameters and solving by linear least squares to obtain a tissue stiffness distribution. The invention also includes a system comprising the components of the DIET system and a computer that is operable to formulate the differential equation model, set up the system of linear equations, and solve the equations by linear least squares to output a tissue stiffness distribution.

The invention further includes a method comprising the steps of providing a set of measured tissue displacements over a region of the tissue; providing a locally homogenous baseline model having an assumed piecewise constant stiffness distribution for the region; formulating a differential equation model describing tissue motion in terms of integrals of the measured breast tissue displacement data; performing a forward simulation using the model to generate a set of model displacements; and comparing the model displacements to the measured tissue displacements to determine if the region contains a tumour.

The invention has the advantages of high efficiency in computation, unique, convex parameter identification, and avoiding very complex finite element forward solver based routines which are highly non-linear, non-convex and starting point dependent. The invention has the flexibility to increase the resolution of space varying patient specific parameters while still maintaining a linear and convex optimization solution with no significant increase in computation.

These and other aspects, objects, features and advantages of the present invention will be more clearly understood and appreciated from a review of the following description of the preferred embodiment and appended claims, and by reference to the accompanying drawings.

### BRIEF DESCRIPTION OF THE DRAWINGS

The present invention is disclosed with reference to the accompanying drawings, wherein:

FIG. 1 is a block diagram of a conventional finite element based parameter identification method.

FIG. 2A is a view of an apparatus implementing the DIET system according to the present invention.

FIG. 2B is a diagram summary of the DIET system.

FIG. 3 is a block diagram of a method, according to the present invention of integral based identification of patient-specific tissue stiffness.

FIG. 4 is a block diagram of a first order differential equation example of a detailed embodiment of the steps of formulating the mathematical model in terms of integrals of measured data and setting up a linear least squares optimization in FIG. 3.

FIG. 5 is an example of a discretized curve analogous to measured data.

FIG. 6 is a block diagram of a one-dimensional Navier-Stokes Equation example describing tissue motion of a detailed embodiment of the steps of formulating the mathematical model in terms of integrals of measured data and setting up a linear least squares optimization in FIG. 3.

FIG. 7 is an output displacement curve for the one-dimensional Navier-Stokes Equation example of FIG. 6.

FIG. 8 is a discretized output displacement curve with 10% noise added for the curve of FIG. 7.

FIG. 9 is the reconstructed stiffness distribution.

FIG. 10 is a block diagram of the two-dimensional Navier-Stokes Equation describing tissue motion of a detailed embodiment of the steps of formulating the mathematical model in terms of integrals of measured data and setting up a linear least squares optimization in FIG. 3.

FIG. 11 is a diagram that shows the discretization of the global domain into a series of elements each with constant stiffness values E_{i,j}, (i, j) ∈ {1,2, . . . 9}.

FIG. 12 is the 2×2 Stencil of FIG. 11 in global coordinates.

FIG. 13 is an arbitrary shape of boundary that is approximated by squares in order to apply the stencil of FIG. 12.

FIG. 14 is the displacement response of the compressible two-dimensional model with a tumour at the (6,5) position in FIG. 11 and 50 Hz actuation.

FIG. 15 is a typical slice through the u displacement surface with 20% uniformly distributed noise based on the median- absolute displacement.

FIG. 16 is the reconstructed stiffness of the two-dimensional domain.

FIG. 17 is the results of identifying a tumour with 20 simulations and varying degrees of noise up to 40%.

FIG. 18 is the results of identifying a tumour for a varying number of points per cm.

FIG. 19 is a block diagram of an alternative method that measures the amount of local homogeneity throughout the domain.

FIG. 20 is a horizontal and vertical slice of the uₘ and vₘ displacements with a tumour placed in the (I,J) position of the global domain of FIG. 11.

FIG. 21 is two curves corresponding to a horizontal slice of the homogenous model simulated displacement uₕ and the measured displacement tₘ.

FIG. 22 is a distance metric used to compare the two curves in FIG. 20.

FIG. 23 is an example of the distance metric over the whole two-dimensional domain before taking a moving average for identifying a 1 cm tumour.

FIG. 24 is an example of the distance metric over the whole two-dimensional domain after taking a moving average for identifying a 1 cm tumour.

FIG. 25 is an example of the distance metric over the whole two-dimensional domain after taking a moving average for identifying a 5 mm tumour.

Corresponding reference characters indicate corresponding parts throughout the several views. The examples set out herein illustrate several embodiments of the invention but should not be construed as limiting the scope of the invention in any manner.

### DETAILED DESCRIPTION

To appreciate the significantly large computational savings and vastly improved accuracy and reliability that integral based parameter identification methods can obtain compared to the current standard non-linear regression, in connection with a DIET system, it is helpful to review the principles and techniques of the non-linear methods.

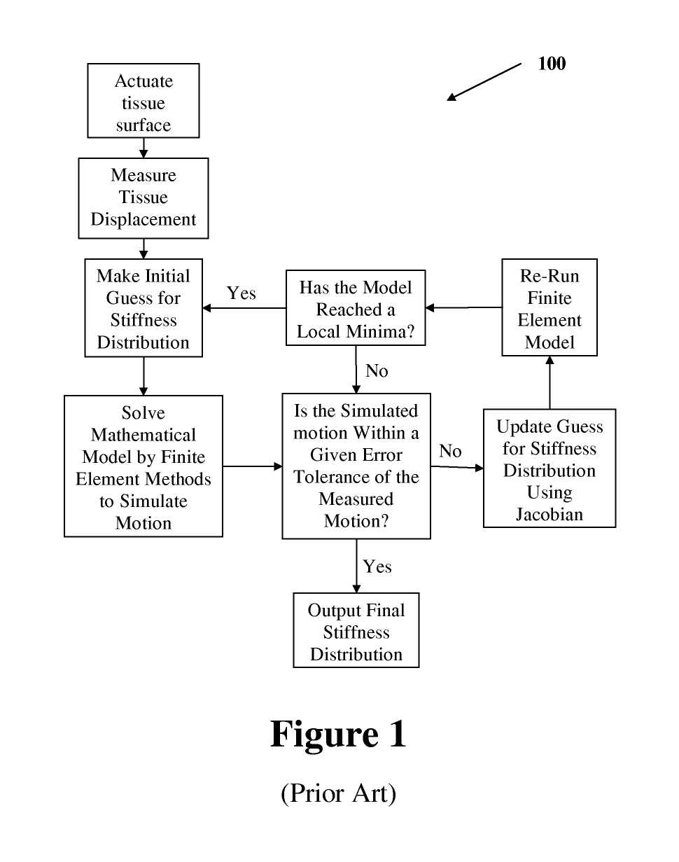

Accordingly, referring to FIG. 1 (prior art) the standard method 100 for identifying unknown parameters in a mathematical model from measured data is shown for the particular case of three-dimensional tissue stiffness reconstruction in the DIET system.

The major computationally expensive step is the process of solving the mathematical model using finite element methods which must be performed many times for each updated guess of the stiffness distribution. The goal is to eventually find a stiffness distribution which minimizes the error between simulated motion and measured motion satisfying some error tolerance. For realistic stiffness distributions and tissue geometries, any finite element method must use potentially 10⁴-10⁶ nodes for sufficient accuracy. Even by breaking the region of interest into smaller sub-regions would require very large computational power only suitable for supercomputers. There is also the problem of converging to the wrong solution, that is, a local minima that can arise from a badly chosen initial stiffness distribution. The conventional solution to this issue is to start at many potentially different starting points, which further significantly increases computational time.

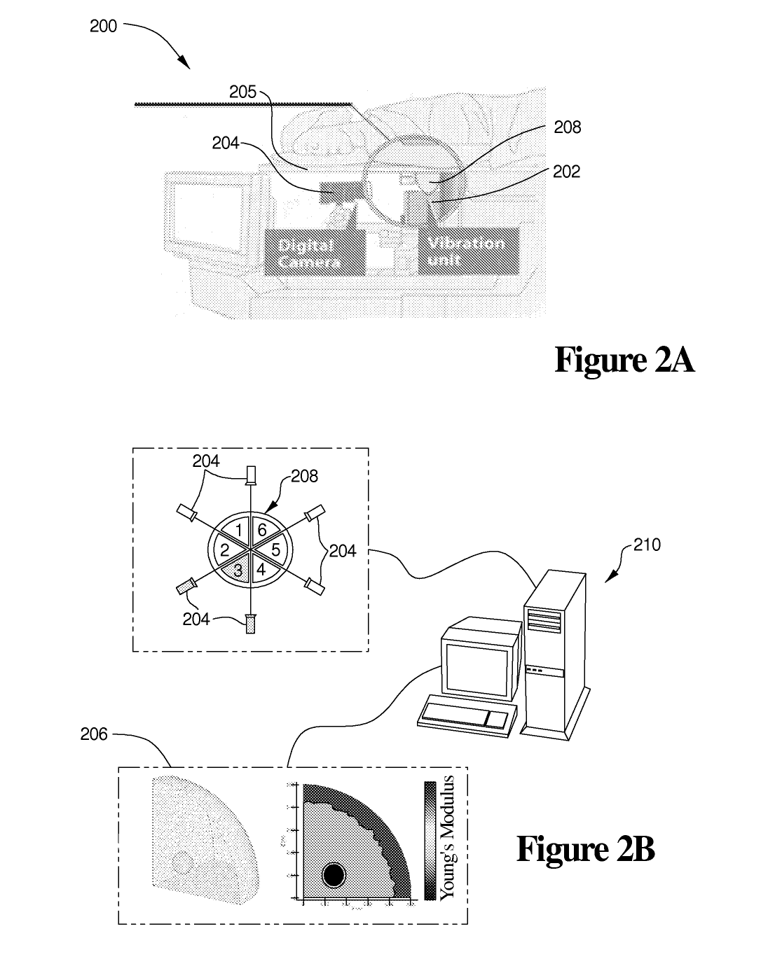

The present invention is generally shown in FIGS. 2A and 213. The invention is herein shown and described for determining the presence of a tumour in breast tissue; however, other tissues may be examined with this method. FIG. 2A shows the apparatus 200 with the patient lying prone on a patient support 205. A vibration unit 202 under the bed/table contacts a breast 208, which is preferably marked with artificial fiducial markings. In an alternative embodiment, two or more vibration units 202 are used to actuate the breast from different locations. An array of cameras 204 below the bed/table captures images of the breast 208 as it is vibrated by the vibration unit 202. The cameras 204 are preferably high resolution cameras, such as those that produce 1 mega-pixel frames. The vibration unit 202 vibrates the breast 208 at a rate that is close to, but offset from the camera speed. For example, the vibration rate would be about 101 Hz for a camera speed of about 100 frames per second. Of course, the camera speed may be a fraction of 100 frames per second and used with the same vibration rate to achieve similar results. The point is to capture a small amount of movement with each frame. The low vibration rates (on the order of 100 Hz) are chosen (as opposed to ultrasonic rates typically used in elasto-tomography) because the breast tissue is much more responsive to vibrations near those frequencies than higher frequencies, such as ultrasonic frequencies.

As shown in FIG. 2B, the cameras 204 are arrayed around the breast 208 and calibrated such that any point on the breast is visible to at least two cameras. The cameras 204 are spatially calibrated by standard techniques used for tracking points with multiple cameras, and the images captured by the cameras are transmitted to the computer 210 for image registration and motion tracking with software to measure the surface motion of the breast 208. Many of the components shown in FIGS. 2A and 2B and described above arc concerned with obtaining the surface motion data of the tissue as described in more detail in the copending application, Global Motion Invariant Signatures For Fast And Accurate Motion Tracking In A Digital Image-Based Elasto-Tomography System, attorney docket number 3023269 US01, the entire disclosure of which is herein incorporated by reference. However, alternative systems and methods for obtaining the measured tissue surface motion data for the present invention of a method for converting the surface motion into a stiffness distribution 206, which may be output in a graphical representation by the computer 210.

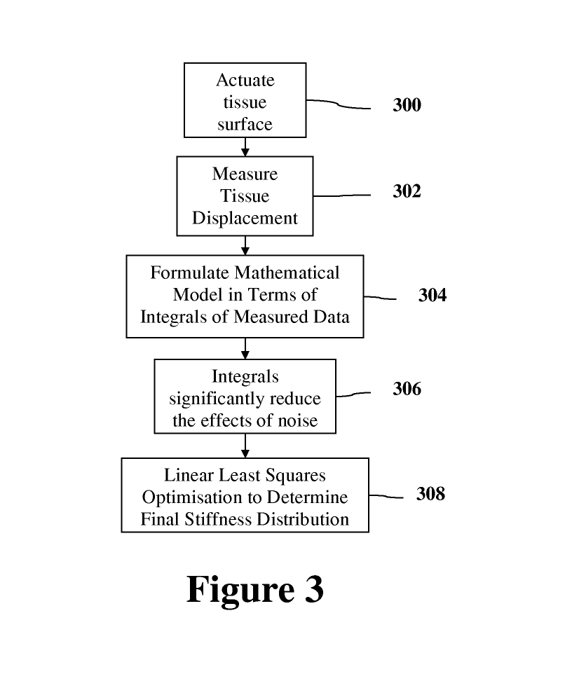

Referring now to FIG. 3, once the tissue is actuated in step 300 and the displacement is measured in step 302 to provide a data set correlating to the surface motion of the tissue, the mathematical model is formulated in terms of integrals of the measured data in step 304, which filter out the noise associated with the measured data (Step 306), and the final stiffness distribution is obtained immediately by linear least squares in step 308. Furthermore, no forward simulations of the mathematical model using the very computationally expensive finite element methods are required.

The integrals by their nature tend to average out the noise associated with the measurements, and thus act as a low pass filter. Other methods of smoothing, for example a Gaussian filter, or simple moving average, distort the measured motion which would result in errors in the measured stiffness. The integral formulation provides a filtering method that is invariant to the tissue motion thus gives a high accuracy in the calculation of three-dimensional stiffness values. This invariance arises from the fact that for small tissue motion the mathematical model given by the Navier-Stokes Equation is an accurate description of the real motion. So by inserting real data into an integral formulation of the model, a set of linear equations in terms of the unknown stiffness values can be set up that accurately describes the tissue motion. The coefficients of the unknown stiffness values are all integrals of the measured noisy data.

Integrals are effectively areas or volumes under curves of surfaces, thus noise has little effect on their numerical values. Also, since the optimisation is now linear, no false solutions corresponding to local minima can occur and it gives greater scope for increasing the resolution of the stiffness distribution to potentially pick up smaller tumours without a significant effect on computation. Furthermore, the inverse construction algorithm could readily be performed on a standard PC.

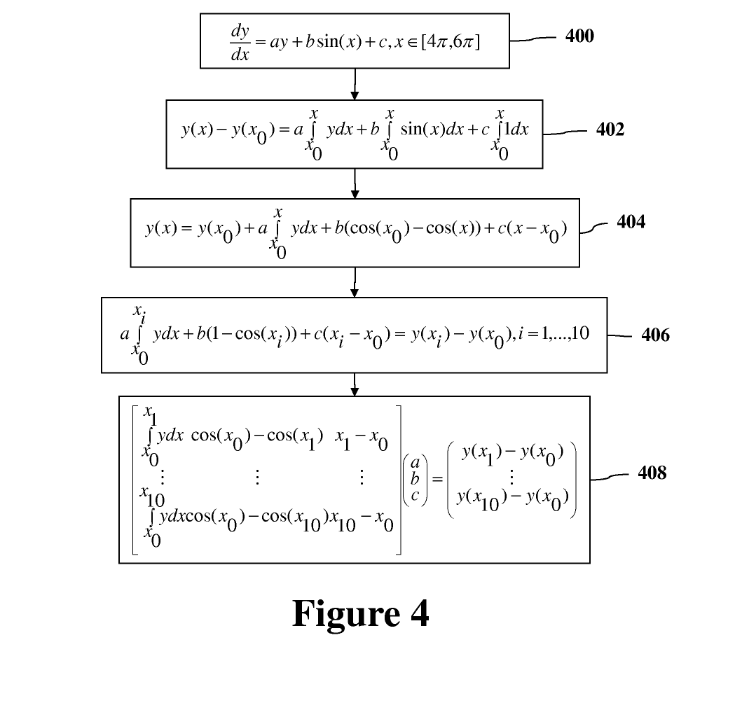

FIG. 4 describes the embodiment of Step 302 and Step 304 in FIG. 3 for the case of a simple first order differential equation given by 400 with three parameters a, b and c. The differential equation 400 describes the motion of a point on the surface of the tissue. 400 is integrated both sides from x₀=4_{π} to x and the parameters a, b, c come outside the integrals producing 402 and 404. 404 is true for all x, so 10 values of x=x₁, . . . , x₀ x ∈ [4π, 6π] are chosen to form an over-determined linear system of 10 equations in 3 unknowns given by 406. 408 is the equivalent matrix system which has a unique solution. Alternatively, one may choose to use more or less values of x for the over-determined linear system of equations so long as the number of values of x chosen is greater than the number of unknowns in the system.

To demonstrate the computational efficiency and accuracy of the integral method compared to the non-linear method, values of a=−0.5, b=−0.2 and c=0.8 are chosen and 400 is solved analytically with an initial condition y(0)=1. The analytical solution is defined:

$$y ( x ) = \frac{1}{( {a}^{2} + 1 )} ( {e}^{ax} ( a + c + ab + {ca}^{2} + {a}^{3} ) - ( ab cos x + {ba}^{2} sin x + {ca}^{2} + c ) ) \qquad ( 1 )$$

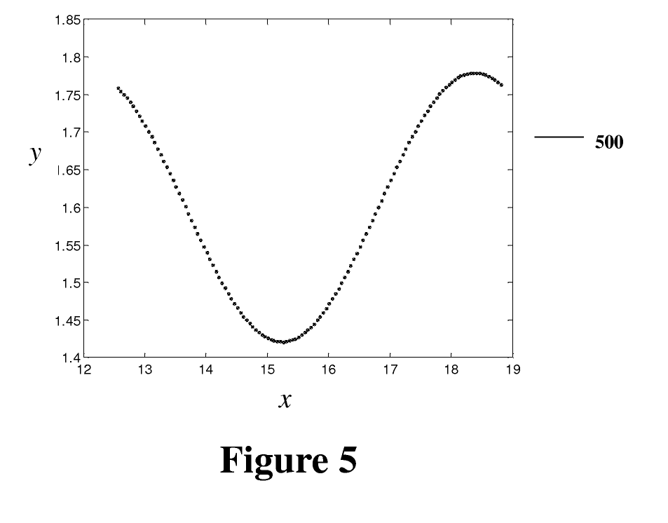

This solution is discretized as shown in the discretized cure 500 of FIG. 5, analogous to measured data. The matrix system 408 is then solved using the trapezium rule to numerically compute the integrals.

The computational time and solutions are shown in Table 1. Also shown is the computational times and the solution for the non-linear method which involves an initial guess for a, b and c in Equation (1) followed by an iterative method of changing a, b and c until the least squares error between Equation (1) and FIG. 5 is minimised.

TABLE 1

| | Starting | CPU Time | |
|---|---|---|---|
| Method | Point | (Seconds) | Solution |
| Integral | — | 0.003 | [−0.5002, −0.2000, 0.8003] |
| Non-Linear | [−1, 1, 1] | 4.6 | [−0.52, −0.20, 0.83] |
| Non-Linear | [1, 1, 1] | 20.8 | [0.75, 0.32, −0.91] |

This is standard non-linear regression. The results show that for the nonlinear method the final solution is highly starting point dependent. That is, the non-linear method can converge to a false solution if a bad starting point is chosen. The integral method found the correct solution immediately and is 1000-10,000 times faster than the non-linear method in this example.

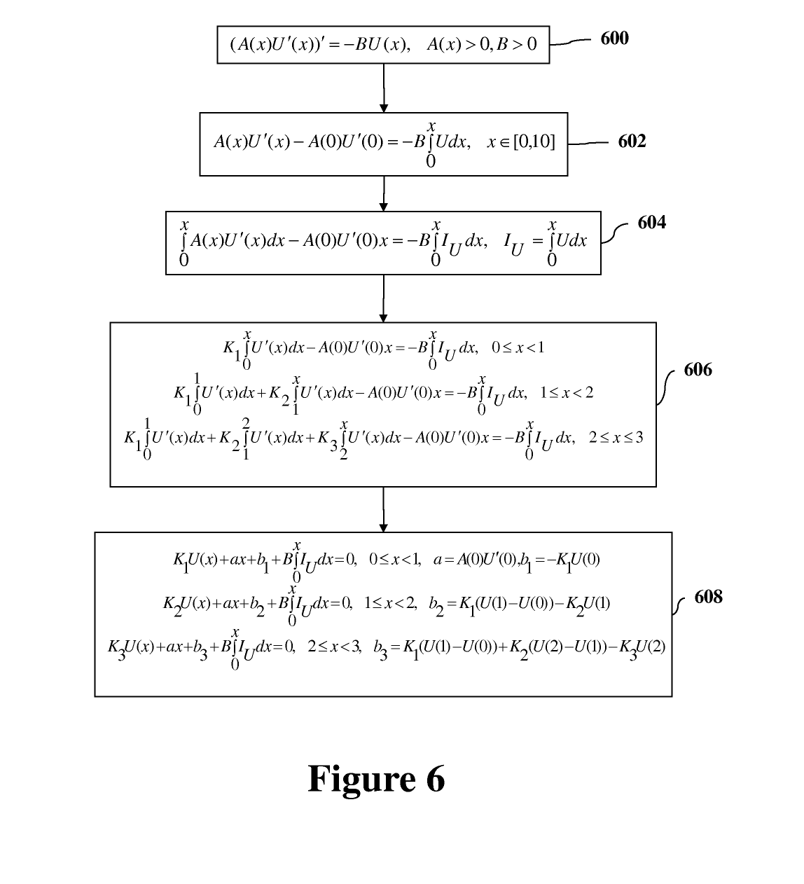

FIG. 6 describes the embodiment of Steps 302 and 304 in FIG. 3 for the case of a second order differential equation 600, which is the one-dimensional form of the Navier-Stokes Equation describing tissue motion. The parameter A(x) describes the stiffness of the tissue and is unknown where the parameter B depends on the density of the tissue and frequency of actuation and is known. For the purposes of illustrations B is chosen to be 5 and:

$$\begin{matrix}A ( x ) = {K}_{1} , 0 \leq x < 1 \\ = {K}_{2} , 1 \leq x < 2 \\ = {K}_{3} , 2 \leq x < 3\end{matrix}$$

where K₁=1, K₂=2, K₃=1 and U(0)=0, U(3)=1.

In this case the results are constrained to be either healthy tissue or tissue corresponding to a tumour wherein A(x)=1 corresponds to healthy tissue, A(x)=2 corresponds to cancerous tissue and U(x) is the tissue displacement. The differential equation 600 is integrated once to give 602 and again to give 604. The equation is then split into three equations in 606 corresponding to the three tissue regions. This is simplified in 608, which is the full integral formulation of 600 with unknown parameters K₁, K₂, K₃, a, b₁, b₂, b₃. Note that a, b₁, b₂, and b₃ are extra parameters added as they depend on values of U at the points x=0, 1, and 2 and U′(0), which could potentially bias the solution because of noise. Making them extra unknowns avoids this possibility.

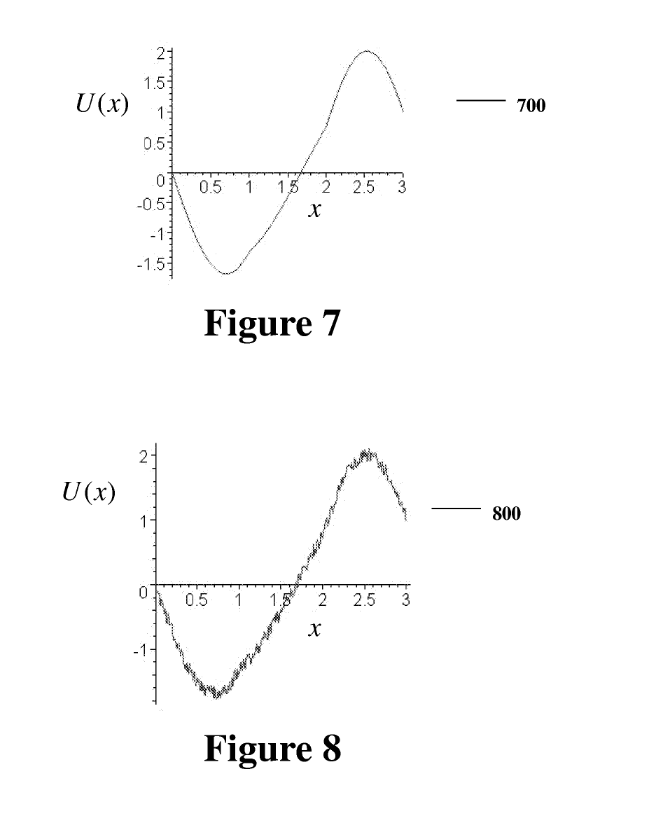

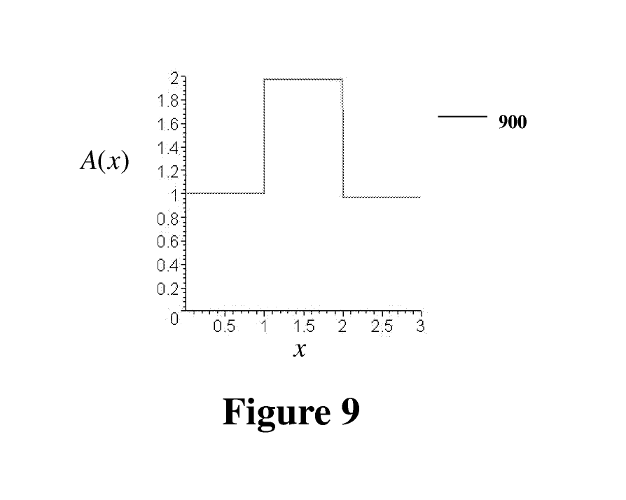

To demonstrate the robustness of the integral method under noise, 600 is solved and shown in the output curve 700 of FIG. 7. 10% uniform noise is then added and shown in the discretized curve 800 of FIG. 8. The displacement U(x) is sampled at 0.01 intervals producing 301 points over the interval [0,3]. Substituting these points into 608 and evaluating the double integrals numerically using the trapezium rule, 301 equations are set in 7 unknowns then solved by linear least squares. The result is given in the output tissue distribution 900 of FIG. 9, which shows that the stiffness value of A(x)=1 and A(x)=2 are recovered very accurately even in the presence of 10% uniformly distributed noise.

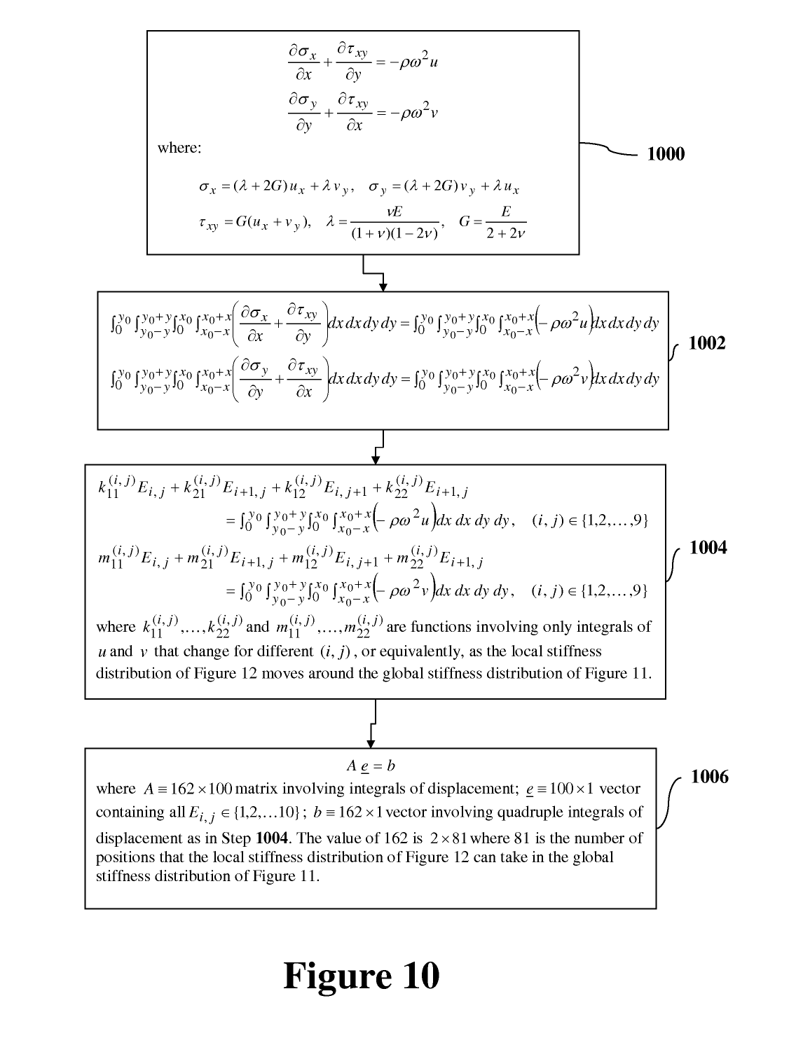

FIG. 10 describes the embodiment of Steps 302 and 304 in FIG. 3 for the case of the compressible two-dimensional Navier-Stokes equations 1000 describing tissue motion. The physical meaning of the parameters in step 1000 are defined as follows:
u, v≡x and y direction displacements (meters; m);
σₓ, σ_{y}≡Longitudinal Stress in the x and v directions (Pascal; Pa);
τ_{xy}≡Shear Stress (Pa); G≡Shear Modulus (Pa); λ≡Lame's Constant;
E≡Young's Modulus (Pa); ν≡Poisson's Ratio;
ρ≡Tissue Density (kilograms per meter cubed; kg·m⁻³);
ω≡Harmonic Actuation Frequency (inverse radians, rads⁻¹).

In this two-dimensional case, four integrals and a special choice of integration limits are required in Step 304 to completely remove the derivatives in the formulation of Step 1000. The integral formulation is shown in Step 1002. The following example shows how a typical second order partial derivative is reduced to only terms involving u and integrals of u:

$$\begin{matrix}{\int }_{0}^{{x}_{0}} {\int }_{{x}_{0} - x}^{{x}_{0} + x} {u}_{xx} ( x , y ) d x = {\int }_{0}^{{x}_{0}} ( {u}_{x} ( {x}_{0} + x , y ) - {u}_{x} ( {x}_{0} - x , y ) ) d x \\ = {\int }_{0}^{{x}_{0}} {u}_{x} ( {x}_{0} + x , y ) d x - \\ {\int }_{0}^{{x}_{0}} {u}_{x} ( {x}_{0} - x , y ) d x\end{matrix} \qquad ( 2 )$$

Applying a substitution z₁=x₀+x and z₂=x₀−x yields a right hand side of Equation (2);

$$\begin{matrix}= {\int }_{{x}_{0}}^{2 {x}_{0}} {u}_{x} ( {z}_{1} , y ) d {z}_{1} - {\int }_{0}^{{x}_{0}} {u}_{x} ( {z}_{2} , y ) d {z}_{2} \\ = ( u ( 2 {x}_{0} , y ) - u ( {x}_{0} , y ) ) - ( u ( {x}_{0} , y ) - u ( 0 , y ) ) \\ = u ( 2 {x}_{0} , y ) - 2 u ( {x}_{0} , y ) = u ( 0 , y )\end{matrix} \qquad ( 3 )$$

Combining the double integral in the left hand side of Equation (2) with the double integral with respect to y eliminates all partial derivatives with respect to both x and y.

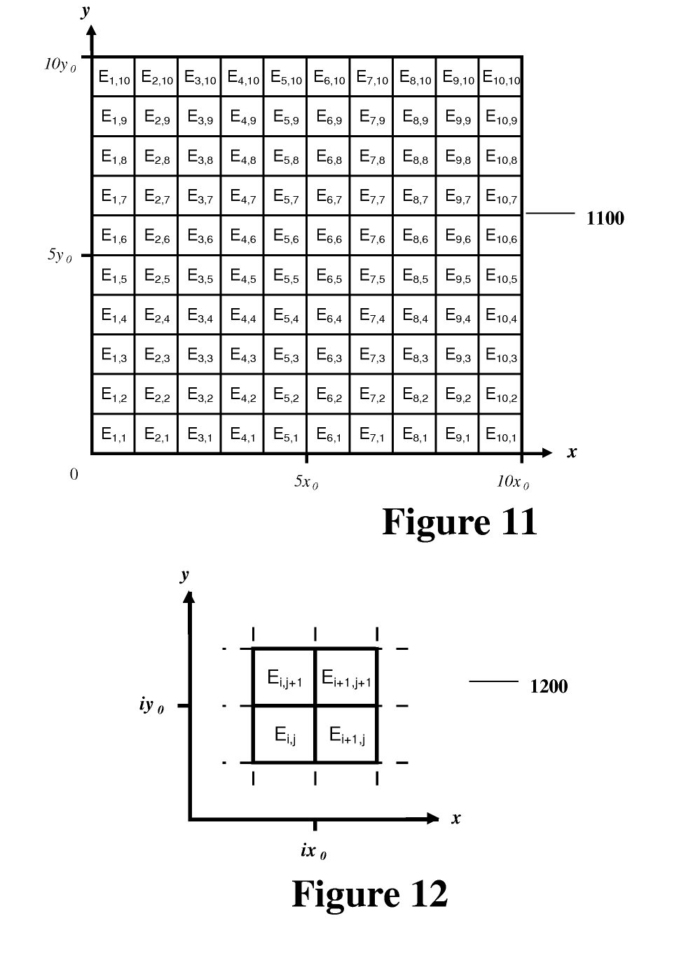

To obtain the final derivative-free formulation corresponding to Step 304, the stiffness distribution E=E(x, y) is assumed to be piecewise constant over the domain of interest. An example of a domain and stiffness distribution 1100 is given in FIG. 11. The simplification of Step 1002 is outlined in Step 1004 and is based on the local stiffness distribution 1200 in FIG. 12, which is varied over the global domain 1100 of FIG. 11. The result is an over-determined system of linear equations in Step 1006 which can be solved by linear least squares; thus determining a piecewise constant approximation to the final output stiffness distribution E(x, y) corresponding to Step 308.

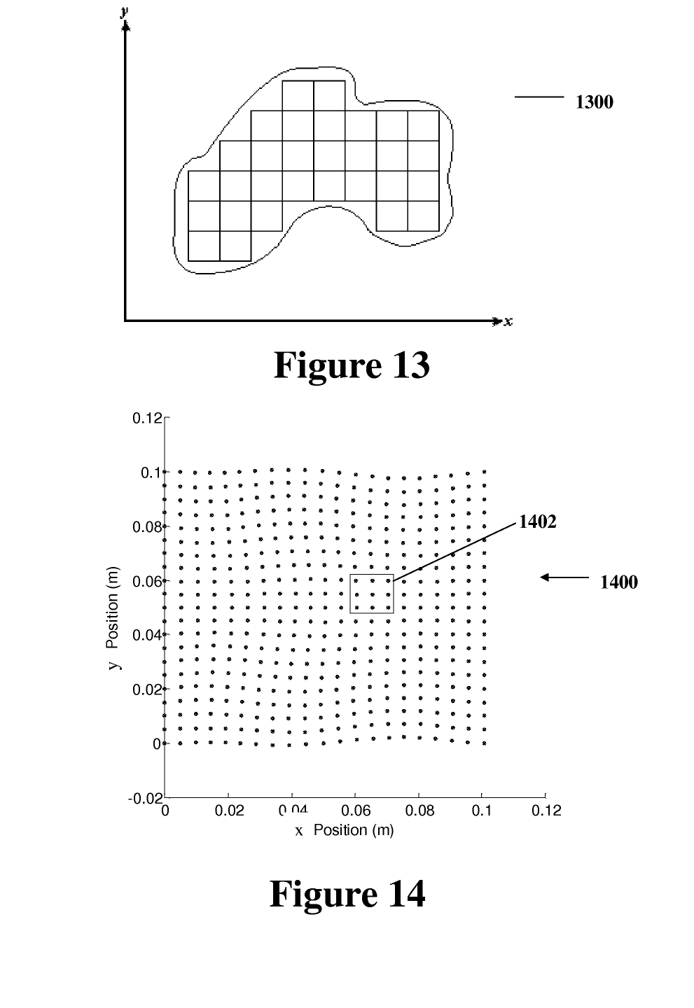

To demonstrate the concept of applying Steps 300-308 in FIG. 3 for the two-dimensional case, the 10 cm×10 cm square domain of FIG. 11 is used to represent the tissue and Young's modulus is taken to be piecewise constant over the 1 cm×1 cm segments. Note that the 2×2 stencil 1200 of FIG. 12 is derived from an equation that makes no assumptions about the boundary conditions applied to the actuated elastic medium. Therefore, the algorithm could be applied to any sinusoidally actuated, two-dimensional plane strain elastic medium with arbitrary boundary conditions and shape. For example, FIG. 13 shows an arbitrary shape 1300 that is first approximated by squares. The stencil of FIG. 12 could then be moved over the domain.

To generate “measured” data for this example, a tumour 10 times stiffer than healthy tissue is placed at the (6,5) position in FIG. 11, which corresponds to the region:

{(x, y):0.06≦x≦0.07, 0.05≦y≦0.06}.

All the surrounding tissue values are held constant at a Young's modulus of 30 kPa representing healthy tissue and Poisson's ratio is taken as ν=0.49, closely representing human tissue.

The tissue is initially actuated at 50 Hz with an amplitude of 1 mm at the right hand boundary of FIG. 11. The left hand boundary is held fixed at zero displacement representing the chest wall. The output displacement response from numerically solving the compressible two-dimensional model of Step 1000 with the appropriate boundary conditions is illustrated in the graph 1400 of FIG. 14 with the 1 cm×1 cm tumour denoted by a square 1402.

However, in the integral formulation of Steps 1002-1006, extensive simulation has shown that the ability to identify a tumour is not significantly affected by increasing Poisson's ratio ν from the value of 0.49; which is the value used to generate the illustrative measured data of Step 302. A value of ν=0.5 corresponds to incompressible tissue, which can be alternatively formulated as zero divergence in the displacement vector field, or uₓ+v_{y}=0. The incompressible two-dimensional Navier-Stokes equations are defined as follows:

$$\begin{matrix}\frac{\partial }{\partial x} ( {Gu}_{x} ) + \frac{\partial }{\partial y} ( {Gu}_{y} ) = - ρ {ω}^{2} u & ( 4 ) \\ \frac{\partial }{\partial x} ( {Gv}_{x} ) + \frac{\partial }{\partial y} ( {Gv}_{y} ) = - ρ {ω}^{2} v & ( 5 )\end{matrix}$$

Equations (4) and (5) can be obtained from the Equations in Step 1000 by the substitution λ=−G. Note that the assumption of λ=−G is only used to reconstruct the stiffness in Step 302; the full two-dimensional compressible Navier-Stokes Equations in Step 1000 are used to generate the illustrative measured data for the example. The major advantage of the incompressibility, or λ=−G assumption, is that the resulting formulation in Steps 1004-1006, is very robust to noise, and only requires a very low data resolution in the measured tissue displacements.

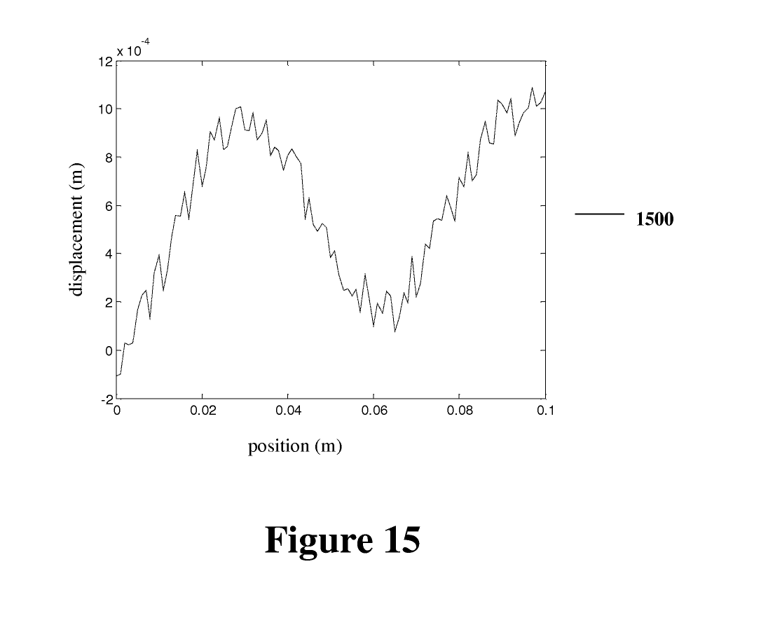

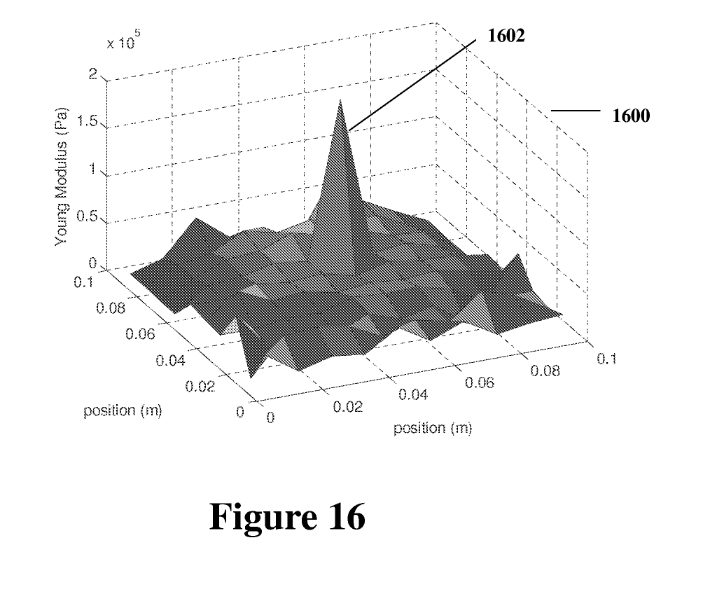

Random uniformly distributed noise is now placed on the displacement data graph 1400 of FIG. 14. The level of noise chosen is based on the median of the absolute displacements. FIG. 15 shows an example curve 1500 of 20% random noise for a typical slice through the u displacement surface. 10 points per cm are taken as the displacement data. Steps 300-308 are applied on the noisy simulated displacements and the resulting reconstructed stiffness curve 1600 is shown in FIG. 16. A large spike 1602 can be seen at the (6,5) position corresponding to the tumour. The height of the spike is 218 kPa, which is 4.3 times stiffer than the highest surrounding healthy tissue, which is 51 kPa and is 7.5 times stiffer than the average surrounding healthy tissue, which is 29 kPa.

Therefore, although the exact stiffness value of 300 kPa (i.e., ten times the value for healthy tissue, which was set at 30 kPa) is not found for the position of the tumour, there is a very significant change in stiffness observed, which is more than sufficient to accurately identify the tumour. The higher than normal healthy tissue value is due to some modelling error from the incompressibility assumption, but this does not effect detection of the tumour.

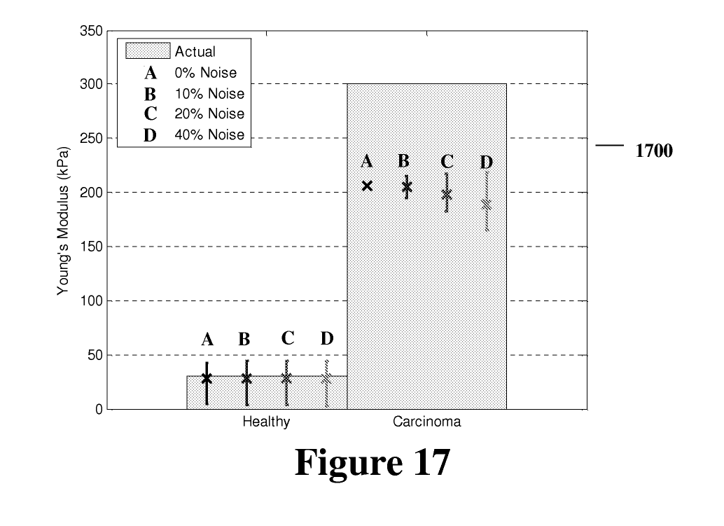

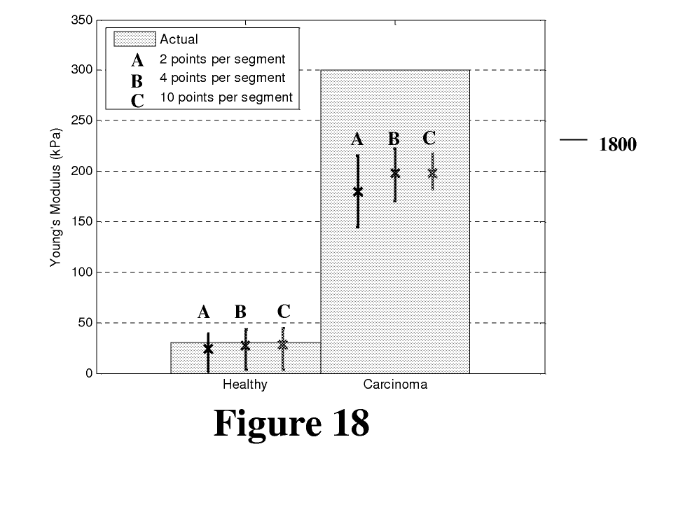

The method of Steps 1002-1006 can also be applied to different boundary conditions) positions of tumour and actuation frequencies. The graph 1700 of FIG. 17 summarizes the results for identifying a tumour at the (7,9) position in FIG. 11, which is actuated at 100 Hz in the vertical direction along the left hand boundary, with all other edges free. 10 points are used per em and the 90% confidence interval about the median value is plotted based on 20 simulations with varying degrees of noise. In all cases with up to 40% noise there is an accurate distinction between healthy tissue and the tumour. For a given amount of 20% noise, the graph 1800 of FIG. 18 gives the results for a varying number of points per cm. Even with on average 2 points per cm or, equivalently, 4 points per cm² there remains a wide separation between the healthy and cancerous tissue.

The accuracy of the method with high noise and low resolution is important as it opens the possibility of cheap, crude and easily portable measurement systems for roughly estimating displacements inside a breast. For example, a simple ultra-sound system could be combined with highly accurate surface measurements from the digital cameras, significantly enhancing the ability to detect tumours.

Note that the stiffness distribution of FIG. 11 assumes the tumour is of a sufficient size (at least 1 cm×1 cm) that part of the tumour is contained in at least one element. If, for example, a 1 cm×1 cm tumour lies halfway between two segments, the stiffness distribution needs to be translated a sufficient number of times horizontally and vertically until most of the cancer is contained in a segment. This process can be done without significantly affecting computation time since the integral-based approach only requires very minimal computation. Also note that to calculate smaller tumours only requires a finer resolution in FIG. 11, which can be readily formulated.

Finally, an alternative concept that will complement the approach of Steps 1002-1006 is presented to allow further flexibility and power to the overall methodology. The method looks to develop a measure of local homogeneity throughout the domain so that cancer will show up as a large error between the measured displacement and the simulated displacement from an assumed locally homogeneous model. In other words instead of directly calculating the stiffness of every segment in the global domain to detect cancer, a low resolution stiffness distribution is assumed throughout the domain, for example a piecewise constant stiffness 3 cm×3 cm elements. This simplified, locally homogeneous model can be rapidly and accurately fitted using the method of Steps 1002-1006 and would readily take into account the natural variations in healthy tissue.

For a given healthy stiffness of 30 kPa, if there is a 300 kPa 1 cm×1 cm tumour present, a 3 cm×3 cm element would only show at best an average stiffness of

(30×8+300)/9=60 kPa,

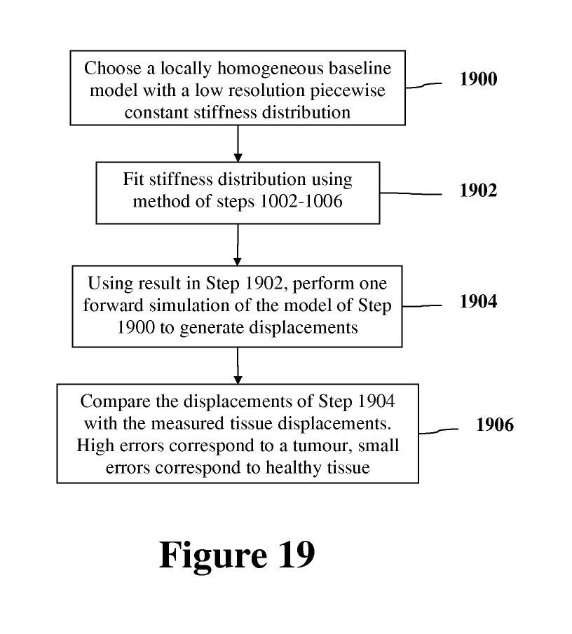

which is borderline between the healthy and cancer range. However, the reconstructed, low resolution, stiffness distribution could he used to generate displacements from one forward simulation of this locally homogeneous model. A region that contains a small tumour will then be highly non-homogeneous, and will thus show up as error between the simulated displacements and the measured displacements. On the other hand, regions containing healthy tissue will have a very good model fit by the forward simulation and thus show significantly smaller error. Steps 1900-1906 of FIG. 19 summarize this method.

To prove the concept of FIG. 19, an example is given where the locally homogeneous model of Step 1900, is taken to be homogeneous over the whole domain. Therefore, one single Young's Modulus is fitted throughout the domain using Steps 1002-1006. The equations and formulation of Step 1002 for this homogeneous case would be similar in structure, but considerably simpler than the non-homogeneous formulation in Steps 1004-1006. The geometry and model parameters of FIG. 14 are used as the basis for testing Steps 1900-1906.

However, in Step 1906, a direct comparison of the homogeneous model generated displacements with the measured data is not sufficient since a tumour has both local effects and global effects on the displacement. Thus the algorithm allows translation of local regions to remove the major global effects and to enhance the local effect of the tumour on its surrounding area.

Let uₘ and vₘ denote the horizontal and vertical measured displacements that result from a tumour placed at some (I,J) position in the global domain of FIG. 1i. Consider a slice of uₘ along the horizontal line L _{y̅}=(x, y̅),0≦x≦0.1) and a slice of vₘ along the vertical line L _{x̅}={( x̅,y),0≦y≦0.1} defined as follows:

X _{y̅}^{(m)}(x)=uₘ(x, y̅),0≦x≦0.1 (6)

Y _{x̅}^{(m)}(y)=vₘ( x̅, y),0≦y≦0.1 (7)

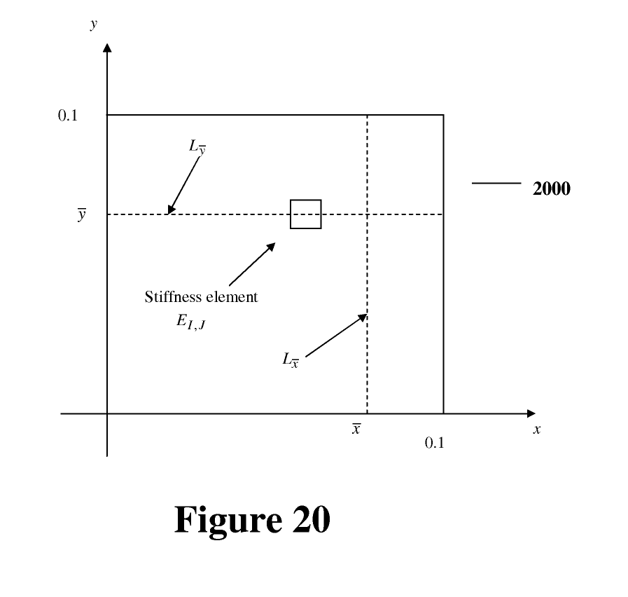

An example of the lines L _{y̅} and L _{x̅} are shown in the graph 2000 of FIG. 20.

Assume that the average stiffness E_{avg} of the global domain of FIG. 11 has been calculated using Steps 1002-1006 based on the measured data uₘ and vₘ. Let uₕ and vₕ denote the horizontal and vertical simulated displacements of the homogeneous model of Step 1904, with a Young's modulus of E=E_{avg}. In a similar way to Equations (6) and (7) define:

X _{y̅}^{(h)}(x)=uₕ(x, y̅), 0≦x≦0.1 (8)

Y _{x̅}^{(h)}(y)=vₕ( x̅, y), 0≦x≦0.1 (9)

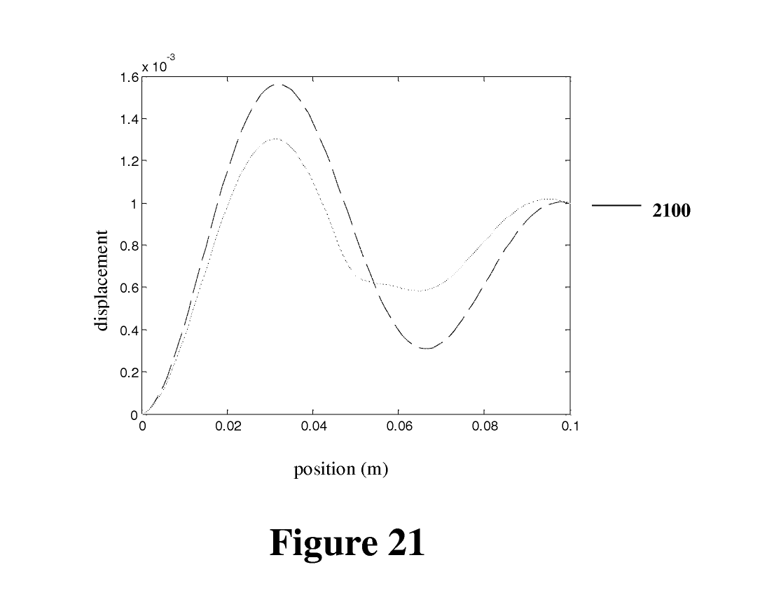

The graph 2100 of FIG. 21 shows an example of the two curves X _{y̅}^{(h)}(x) (dashed) and X _{y̅}^{(m)}(x) (solid) corresponding to Equations (6) and (8) overlaid. For this example, the model of FIG. 14 is used, except the tumour is placed at the (5,6) position in the global domain of FIG. 11. The horizontal displacement in the x direction and vertical displacement in the y direction are chosen as they have been found to most consistently characterize changes in motion due to a tumour.

As can be seen in FIG. 21, the greatest change in topology of X _{y̅}^{(m)}(x) as compared to X _{y̅}^{(h)}(x) occurs between x=0.05 and 0.06 corresponding to the position of the cancer. However, before directly analysing the difference X _{y̅}^{(m)}(x)−X _{y̅}^{(h)}(x), each curve is reformulated to take into account topologically similar regions of the two curves that may be related by a translation. A discretization of 10 equally spaced points per cm is used for the x coordinate and denoted by {xᵢ, i=0, . . . , 100} , where x₀=0 and x₁₀₀=0.1. Similarly the discretization of the y coordinate is denoted by {yᵢ, i=0, . . . , 100} , where y₀=0 and y₁₀₀=0.1. To successfully locate the cancer a distance metric is defined between the curves X _{y̅}^{(m)}(x) and X _{y̅}^{(h)}(x) in Equations (6) and (8) as follows:

$$\begin{matrix}{D }_{\overline{y}}^{X} ( {x}_{i} ) = {‖ {\hat{X} }_{\overline{y}}^{( m )} ( {x}_{i} ) - {\hat{X} }_{\overline{y}}^{( h )} ( {x}_{i} ) ‖}_{2} , i = 5 , … , 95 \\ \overline{y} ∈ \{ {y}_{1} , … {y}_{99} \} \\ where \text{:} & ( 10 ) \\ {\hat{X}}_{\overline{y}}^{( m )} ( {x}_{i} ) = {X}_{\overline{y}}^{( m )} ( {x}_{i} ) - mean \{ {X}_{\overline{y}}^{( m )} ( {x}_{i - 5} ) , … , {X}_{\overline{y}}^{( m )} ( {x}_{i + 5} ) \} & ( 11 ) \\ {\hat{X}}_{\overline{y}}^{( h )} ( {x}_{i} ) = {X}_{\overline{y}}^{( h )} ( {x}_{i} ) - mean \{ {X}_{\overline{y}}^{( h )} ( {x}_{i - 5} ) , … , {X}_{\overline{y}}^{( h )} ( {x}_{i + 5} ) \} & ( 12 ) \\ \begin{matrix}{D}_{\overline{y}} ( {x}_{i} ) = {D}_{\overline{y}} ( {x}_{5} ) , i = 0 , … , 4 \\ = {D}_{\overline{y}} ( {x}_{95} ) , i = 96 , … , 100\end{matrix} & ( 13 )\end{matrix}$$

Note that the mean in Equations (11) and (12) is essentially a moving 11 point average. Subtracting the moving mean in Equations (11) and (12) ensures that if parts of X _{y̅}^{(m)}(x) and X _{y̅}^{(h)}(x) are related by a vertical translation, the corresponding parts of \hat{X} _{y̅}^{(m)}(x) and \hat{X} _{y̅}^{(h)}(x) will be identical. Otherwise, a vertical translation could add extra unwanted error to the norm in Equation (10) which may hide the position of the tumour. For simplicity in the formulation, a horizontal translation was not considered. In simulation this assumption has been shown to not effect the detection of the tumour.

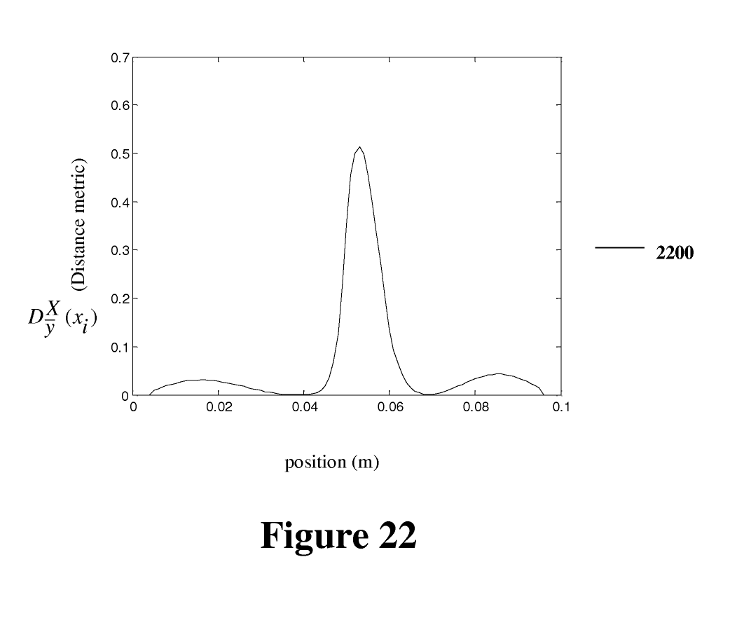

The curve 2200 of FIG. 22 shows the result of plotting the 2-norm in Equation (10), where the tumour is clearly revealed at the maximum value of the 2-norm across the whole interval.

Similarly to Equations (10)-(13), a distance metric is defined between the curves Y _{y̅}^{(m)}(y) and Y _{y̅}^{(h)}(y) in Equations (7) and (9) as follows:

$${D}_{\overline{x}}^{Y} ( {y}_{j} ) = {‖ {\hat{Y}}_{\overline{y}}^{( m )} ( {y}_{j} ) - {Y}_{\overline{y}}^{( h )} ( {y}_{j} ) ‖}_{2} , j = 5 , … , 95 \\ \overline{x} ∈ \{ {x}_{1} , … , {x}_{99} \} \qquad ( 14 )$$

where:

Ŷ _{y̅}^{(m)}(xᵢ)=Y _{x̅}^{(m)}(yⱼ)−mean{Y _{x̅}^{(m)}(yᵢ₋₅), . . . , Y _{x̅}^{(m)}(yᵢ₊₅)}(15)

Ŷ _{y̅}^{(h)}(xⱼ)=Y _{x̅}^{(h)}(yⱼ)−mean{Y _{x̅}^{(h)}(yᵢ₋₅), . . . , Y _{x̅}^{(h)}(yᵢ₊₅)}(16)

$$\begin{matrix}{D}_{\overline{x}} ( {y}_{j} ) = {D}_{\overline{x}} ( {y}_{5} ) , j = 0 , … , 4 \\ = {D}_{\overline{x}} ( {y}_{95} ) , j = 96 , … , 100\end{matrix} \qquad ( 17 )$$

A distance metric combining Equations (10) and (14) is now defined to enhance the detection of cancer:

$$D ( {x}_{i} , {y}_{j} ) = \frac{{D}_{{y}_{j}}^{X} ( {x}_{i} ) {D}_{{x}_{i}}^{Y} ( {y}_{j} )}{{u}_{median}^{( m )} {v}_{median}^{( m )}} , ( i , j ) ∈ \{ 1 , … , 99 \} \qquad ( 18 )$$

where u_{median}^{(m)} and v_{median}^{(m)} are the median absolute values of the measured displacements of uₘ and vₘ, which are used to ensure the metric is approximately on the same scale as the measured data. Equation (18) defines a smooth surface over the global domain of FIG. 11 and is effectively a measure of the degree of local homogeneity surrounding each point (xᵢ, yⱼ). The more homogeneous the region surrounding (xᵢ, yⱼ) the smaller the value of D(xᵢ, yⱼ).

In summary, Equations (6)-(18) are used to compare the displacements of Step 1904 with the measured tissue displacements, thus completing the final Step 1906.

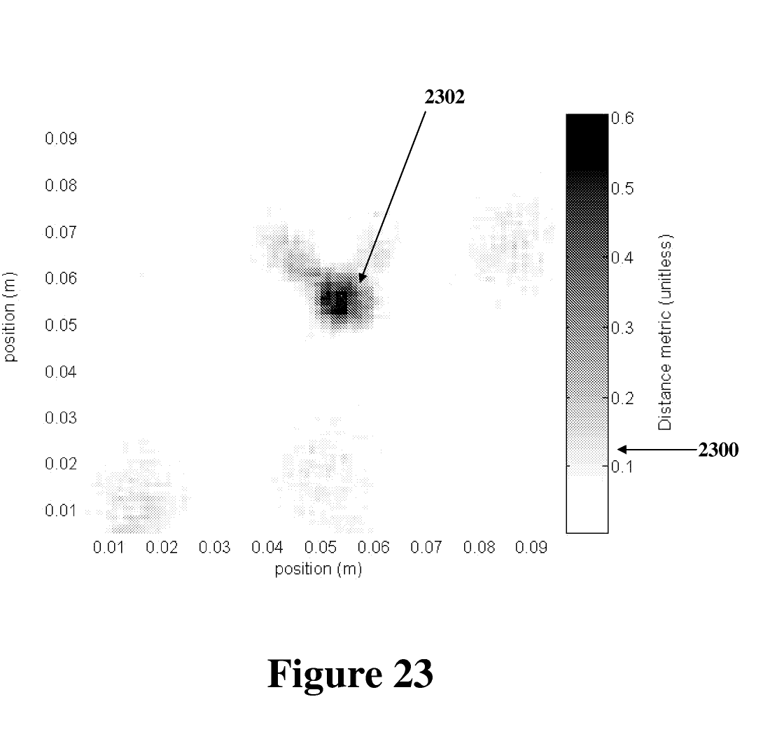

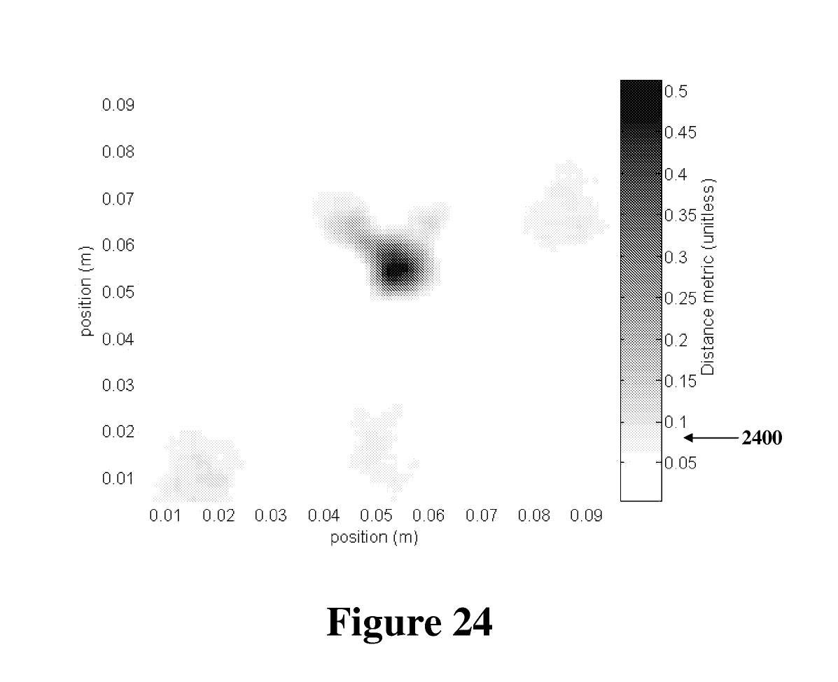

The graph 2300 of FIG. 23 shows the distance metric D(xᵢ, yⱼ) of Equation (18) over the whole domain calculated from Equations (6)-(18) for a similar example to the model of FIG. 14 except the tumour is placed at the (5,6) position in the global domain of FIG. 11. The actuation frequency is 50 Hz which is the same as that in FIG. 14 and 100% random uniformly distributed noise is added. As can be seen in FIG. 23, the 1 cm tumour 2302 is clearly identified. However a further improvement in the clarity of FIG. 23 can be made by applying one simple 9 point moving average over the two-dimensional data set. For a relatively coarse grid of 10 points per cm this has negligible effect on computation. The graph 2400 of FIG. 24 shows that after applying this moving average the tumour is even more enhanced.

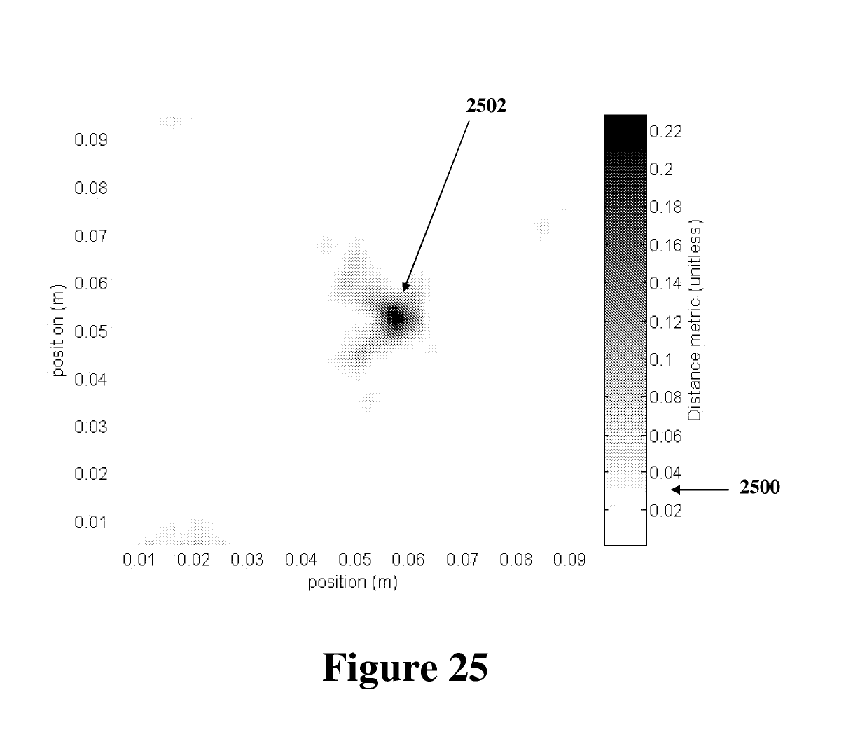

To further demonstrate this method, a 5 mm×5 mm tumour is placed at the position 0.056≦x≦0.061, 0.05≦y≦0.055 and Steps 1900-1906 are applied. The result is shown in the graph 2500 of FIG. 25, again demonstrating an accurate identification of the tumour. Thus the algorithms of FIGS. 10 and 19 are very effective at identifying a tumour 2502 in the case of the two-dimensional Navier-Stokes Equations.

Finally, the same approach as outlined above for the two-dimensional case can be applied to the full three-dimensional compressible Navier-Stokes Equations defined:

$$\begin{matrix}\frac{\partial }{\partial x} ( G {u}_{x} ) + \frac{\partial }{\partial y} ( G {u}_{y} ) + \frac{\partial }{\partial z} ( G {u}_{z} ) + \frac{\partial }{\partial x} [ ( λ + G ) ( \frac{\partial u}{\partial x} + \frac{\partial v}{\partial y} + \frac{\partial w}{\partial z} ) ] = - ρ {ω}^{2} u & ( 20 ) \\ \frac{\partial }{\partial x} ( G {v}_{x} ) + \frac{\partial }{\partial y} ( G {v}_{y} ) + \frac{\partial }{\partial z} ( G {v}_{z} ) + \frac{\partial }{\partial y} [ ( λ + G ) ( \frac{\partial u}{\partial x} + \frac{\partial v}{\partial y} + \frac{\partial w}{\partial z} ) ] = - ρ {ω}^{2} v & ( 21 ) \\ \frac{\partial }{\partial x} ( G {w}_{x} ) + \frac{\partial }{\partial y} ( G {w}_{y} ) + \frac{\partial }{\partial z} ( G {w}_{z} ) + \frac{\partial }{\partial z} [ ( λ + G ) ( \frac{\partial u}{\partial x} + \frac{\partial v}{\partial y} + \frac{\partial w}{\partial z} ) ] = - ρ {ω}^{2} w & ( 22 )\end{matrix}$$

Equations (20)-(22) can be reformulated by using six integrals over the coordinates x, y and z;

$${\int }_{0}^{{z}_{0}} {\int }_{{z}_{0} - z}^{{z}_{0} + z} {\int }_{0}^{{y}_{0}} {\int }_{{y}_{0} - y}^{{y}_{0} + y} {\int }_{0}^{{x}_{0}} {\int }_{{x}_{0} - x}^{{x}_{0} + x} \qquad ( 23 )$$

which are of the same form as the four integrals in Step 1002. Furthermore, the incompressibility condition can be readily applied in the three-dimensional case by again setting the divergence to zero, uₓ+v_{y}+w_{z}=0 resulting in the following equations:

$$\begin{matrix}\frac{\partial }{\partial x} ( G {u}_{x} ) + \frac{\partial }{\partial y} ( G {u}_{y} ) + \frac{\partial }{\partial z} ( G {u}_{z} ) = - ρ {ω}^{2} u & ( 24 ) \\ \frac{\partial }{\partial x} ( G {v}_{x} ) + \frac{\partial }{\partial y} ( G {v}_{y} ) + \frac{\partial }{\partial z} ( G {v}_{z} ) = - ρ {ω}^{2} v & ( 25 ) \\ \frac{\partial }{\partial x} ( G {w}_{x} ) + \frac{\partial }{\partial y} ( G {w}_{y} ) + \frac{\partial }{\partial z} ( G {w}_{z} ) = - ρ {ω}^{2} w & ( 26 )\end{matrix}$$

As with the two-dimensional case, substituting λ=−G into the full compressible three-dimensional Navier-Stokes equations results in Equations (24)-(26). The integrals of Equation (19) remove all derivatives in both the compressible and incompressible three-dimensional Navier-Stokes equations. The incompressible form of λ=−G is then used to reconstruct stiffness by setting up an over-determined system of linear equations and solving by linear least squares.

While the invention has been described in terms of steady state motion, one will note that the tissue may be actuated at a frequency that varies with time, an amplitude that varies with time, or both. In these non-steady state cases, the Navier-Stokes equations will include a time component and the process will include the integral of the equations with respect to time as well as space before conducting the linear least squares analysis.

While the invention has been described with reference to preferred embodiments, it will be understood by those skilled in the art that various changes may be made and equivalents may be substituted for elements thereof to adapt to particular situations without departing from the scope of the invention. Therefore, it is intended that the invention not be limited to the particular embodiments disclosed as the best mode contemplated for carrying out this invention, but that the invention will include all embodiments falling within the scope and spirit of the appended claims.

### PARTS LIST

100 standard finite element based method
200 apparatus
202 vibration unit
204 array of calibrated digital cameras
205 patient support
206 stiffness distribution of the tissue
208 the tissue, particularly a breast
210 computer system
300 actuation of tissue surface
302 tissue displacement measurement
304 integral formulation
306 low pass filter effect of the integrals
308 linear least squares methodology to determine final stiffness distribution
400 first order differential equation example
402 first integral formulation
404 second integral formulation
406 forming a system of integral equations
408 linear least squares system of over-determined equations
500 discretized curve
600 one-dimensional Navier-Stokes equation
602 integrating once
604 integrating a second time
606 setting up integral equations
608 simplifying integral equations
700 output curve from one-dimensional Navier-Stokes equation
800 discretized curve with 10% random noise
900 output tissue distribution
1000 two-dimensional compressible Navier-Stokes equations
1002 integrating four times
1004 formulating integral equations
1006 setting up linear equations in the stiffness elements
1100 global domain with constant piecewise stiffness distribution
1200 local 2×2 stencil used to generate over-determined system of linear equations
1300 arbitrary boundary shape approximated by squares
1400 simulated tissue displacement with tumour
1402 simulated tumour
1500 curve showing random uniformly distributed noise added to displacements
1600 curve showing distance metric to detect cancer
1602 spike indicating a tumour
1700 graph demonstrating separation between Carcinoma and Healthy tissue for varying degrees of noise
1800 graph demonstrating separation between Carcinoma and Healthy tissue for a varying number of points per cm
1900 choice of baseline model and stiffness distribution
1902 fitting stiffness distribution
1904 performing one forward simulation
1906 comparing topologically the differences between simulated and measured displacements
2000 graph showing horizontal and vertical slices of the displacement
2100 graph overlaying homogenous model generated curve and measured curve containing a tumour
2200 curve showing distance metric
2300 graph revealing 1 cm tumour without moving average
2302 1 cm tumour
2400 graph revealing 1 cm tumour with moving average
2500 graph revealing 5 mm tumour with moving average
2502 5 mm tumour

1. A method for obtaining patient specific parameter identification with a minimal amount of computation in connection with a digital image-based elasto-tomography system, said method comprising the steps of:
(a) actuating a tissue;
(b) tracking the surface motion of the tissue to provide measured tissue displacement data;
(c) formulating a differential equation model describing tissue motion in terms of integrals of the measured breast tissue displacement data;
(d) setting up a system of linear equations in the space varying tissue stiffness parameters and solving by linear least squares to obtain a tissue stiffness distribution.

2. The method of claim 1 wherein the differential equation model describes steady state motion.

3. The method of claim 1 wherein the differential equation model describes non-steady state motion.

4. The method of claim 3 wherein the integrals are with respect to time and space.

5. The method of claim 3 wherein the measured tissue displacement data was obtained by actuating the tissue at a constant frequency and a constant amplitude.

6. The method of claim 1 wherein the differential equation model is assumed to be incompressible.

7. The method of claim 1 further comprising the step of using a low resolution, space varying tissue distribution to initially describe an assumed healthy tissue model wherein a region corresponds to a tumour where a topological shape of the simulated healthy model displacements differs a predetermined amount from the measured data.

8. The method of claim 1 wherein the space varying tissue stiffness parameters are constant piecewise elements over a domain and a class of various alignments are chosen.

9. The method of claim 1 wherein the tissue is actuated with a time-varying frequency.

10. The method of claim 1 wherein the tissue is actuated with a time-varying amplitude.

11. The method of claim 1 further comprising the step of applying constraints on the stiffness values in the stiffness distribution.

12. The method of claim 11 wherein the stiffness values are constrained to lie in one of a healthy range and a tumor range.

13. The method of claim 11 wherein the constraints are that no high stiffness elements are allowed.

14. The method of claim 11 where all stiffness values are assumed to be substantially equal.

15. The method of claim 11 wherein the stiffness values are constrained to one group of high stiffness elements.

16. The method of claim 11 wherein the tissue is breast tissue.

17. A apparatus for obtaining patient specific parameter identification with a minimal amount of computation in connection with a digital image-based elasto-tomography system, comprising:
a motion sensor having a field of view;
a tissue vibration unit for vibrating tissue of a patient; and
a computer system in electrical communication with the motion sensor and being operable to record and compute the surface motion of tissue actuated by the vibration unit and within the field of view of the motion sensor, and output the measured tissue surface motion;
wherein the computer system is operable to formulate a differential equation model describing tissue motion in terms of integrals of the measured tissue surface motion, set up a system of linear equations in the space varying tissue stiffness parameters, and solve the equations by linear least squares to obtain a unique patient specific tissue stiffness distribution.

18. The apparatus of claim 17, further comprising a patient support proximate to the motion sensor and the vibration unit.

19. The apparatus of claim 17, the motion sensor comprising an array of spatially calibrated cameras.

20. The apparatus of claim 17, the computer running a software package to determine the stiffness distribution of the tissue.

21. A method for obtaining patient specific parameter identification with a minimal amount of computation in connection with a digital image-based elasto-tomography system, said method comprising the steps of:
(a) actuating a tissue;
(b) tracking the surface motion of the tissue to provide a set of measured tissue displacement data over a region of the tissue;
(c) providing a locally homogenous baseline model having an assumed piecewise constant stiffness distribution for the region;
(d) formulating a differential equation model describing tissue motion in terms of integrals of the measured breast tissue displacement data;
(e) performing a forward simulation using the model to generate a set of model displacements; and
(f comparing the model displacements to the measured tissue displacements to determine if the region contains a tumour.

22. The method of claim 21, wherein a significant difference between the model displacements and the measured tissue displacements corresponds to a tumour.

23. The method of claim 21, wherein the model has a low resolution relative to the potential size of a tumour.
